(19) United States
(12) Patent Application Publication
(10) Pub. No.: US 2023/0233452 A1
(43) Pub. Date: Jul. 27, 2023

(54) Topical Lip Balm Composition and Method of Use Thereof for Healing Cold Sores

(71) Applicant: Allenoush Beard, Summerfield, ND (US)

(72) Inventor: Allenoush Beard, Summerfield, ND (US)

(21) Appl. No.: 17/865,546

(22) Filed: Jul. 15, 2022

Related U.S. Application Data
(60) Provisional application No. 63/301,699, filed on Jan. 21, 2022.

(51) Int. Cl. A61K 9/00; A61K 47/44; A61K 31/045; A61K 33/38

(52) U.S. Cl. CPC A61K 9/0014; A61K 47/44; A61K 31/045; A61K 33/38

(57) ABSTRACT

The present invention relates to an easy to administer topical lip balm/ointment composition for quickly healing fever blisters caused by the HSV-1 virus. The lip balm/ointment is topically applied to an affected area using a fingertip, or rolling a composition stick, and is penetrated deep into the skin for blocking the virus and protecting healthy skin cells. The composition includes a unique formulation of natural and organic ingredients consisting of beeswax, shea butter, liquid coconut oil, organic lemon or peppermint flavor, menthol, and colloidal silver. The composition heals the cold sore within 48 hours without using any harmful chemicals available in over-the-counter pharmacy products. The composition is available in small containers or rolling sticks.

### CROSS-REFERENCE TO RELATED APPLICATION

The present application claims priority to, and the benefit of, U.S. Provisional Application No. 63/301,699, which was filed on Jan. 21, 2022 and is incorporated herein by reference in its entirety.

### FIELD OF THE INVENTION

The present invention relates generally to the field of treating fever blisters or cold sores caused by the HSV-1 virus or other ailments. More specifically, the present invention relates to a novel lip balm/ointment composition that includes topical application and a unique combination of natural and organic ingredients, and weight percentages thereof, for quickly healing cold sores (fever blisters) in 24 to 48 hours. The composition does not contain any harmful chemicals and therefore can be used by individuals of all ages. The composition is topically applied and can be penetrated into the cold sore affected skin of a user. Accordingly, the present disclosure makes specific reference thereto. Nonetheless, it is to be appreciated that aspects of the present invention are also equally applicable to other like applications, devices, and methods of manufacture.

### BACKGROUND

By way of background, a cold sore is a group of tiny, painful blisters that can be caused by the herpes simplex virus-1 (HSV-1) and other viruses. Cold sores can also be called fever blisters or herpes simplex labialis. Research shows that up to 50%-80% of individuals in the U.S. have at least one form of HSV. Cold sores generally occur on lips and affected areas and often become red, swollen, and painful. The blisters may also break open and fluid may seep therefrom. Individuals can get HSV-1 by kissing or touching someone with cold sores, or by sharing eating utensils, towels, or other items that have been used by an infected person.

The conventional healing period for blisters is generally about seven to fourteen days, which is a long time to be in pain before the blister heals. Individuals use cold compresses, prescriptions, or over-the-counter pharmacy treatments, such as, antiviral topical treatments to treat the blisters. Many individuals take pain killers for easing pain, but such method does not help in shortening the healing time of cold sores. Further, such over-the-counter pharmacy treatments and products can have chemicals that can be harmful for users.

HSV-1 is contagious and individuals having cold sores, upon touching their eyes, may cause damage to the eyes as HSV-1 may enter the eyes. Individuals desire an improved composition that can be topically applied to cold sores to shorten healing time and discomfort due to blisters.

Therefore, there exists a long-felt need in the art for a composition that can be topically applied to blisters caused by HSV-1 for shortening healing time. There is also a long-felt need in the art for an improved lip balm/ointment composition that includes organic and natural ingredients and that can be used for healing cold sores. Additionally, there is a long-felt need in the art for a base composition that quickly reduces discomfort, pain, and redness of cold sore blisters. Moreover, there is a long-felt need in the art for a base composition that can be topically applied to the affected area on lips and adjacent skin, and can penetrate lip skin. Further, there is a long-felt need in the art for an improved lip balm/ointment that not only soothes the lips but also helps in quickly healing fever blisters caused by the HSV-1 virus. Finally, there is a long-felt need in the art for a base composition that offers a simple method of treating cold sores while significantly shortening healing time without using any harmful chemicals.

The subject matter disclosed and claimed herein, in one embodiment thereof, comprises a topical base composition for topical application as an organic lip balm/ointment on fever blisters and cold sores caused by HSV-1. The composition consisting of (i) 23.00 weight percentage beeswax; (ii) 23.00 weight percentage organic shea butter; (iii) 7.70 weight percentage organic coconut oil; (iv) 30.90 weight percentage organic lemon flavoring agent; (v) 7.70 weight percentage menthol; and (vi) 7.70 weight percentage 500 ppm colloidal silver, wherein the composition helps in treating fever blisters caused by the HSV-1 virus within 48 hours. The composition remains solid/semi-solid at room temperature and can be stored in containers from about 1 oz. to about 4 oz. in volume.

In this manner, the lip balm/ointment composition of the present invention accomplishes all of the foregoing objectives and provides users with improved lip balm/ointment for treating fever blisters and cold sores caused by the HSV-1 virus. The composition has natural and organic ingredients and therefore, prevents users from having to use over the counter pharmacy products containing harmful chemicals. The healing time of blisters can be shortened to within 48 hours rather than the typical seven-to-fourteen-day healing time.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a topical base composition in a solid or semi-solid form for applying as an organic lip balm/ointment. The composition comprising a plurality of organic ingredients, consisting of (i) beeswax; (ii) shea butter; (iii) liquid coconut oil; (iv) organic flavoring agent; (v) menthol; and (vi) colloidal silver, each in amounts that are effective for collectively forming a topical base composition having an ability to topically apply as a lip balm/ointment, wherein the composition is effective in penetration of lip skin and cold sores for treating fever blisters caused by the HSV-1 virus.

In yet another embodiment, a topical lip balm/ointment composition is disclosed. The lip balm/ointment composition comprising of natural and organic ingredients for healing cold sores within 48 hours and consisting of (i) 10-30 weight percentage beeswax; (ii) 10-30 weight percentage organic shea butter; (iii) 5-20 weight percentage organic coconut oil; (iv) 15-35 weight percentage organic flavoring agent (i.e. lemon, peppermint, et. al.); (v) 2-15 weight percentage menthol; and (vi) 1-40 weight percentage 500 ppm colloidal silver, wherein the composition has an ability to function as a topically applied vehicle for ingredients and helps in treating fever blisters and cold sores caused by the HSV-1 virus.

In yet another embodiment, a solid topical lip balm/ointment composition is disclosed. The lip balm/ointment, in one exemplary composition, comprising of natural and organic ingredients for treating fever blisters within forty-eight hours and consisting of (i) 23% w/w beeswax; (ii) 23% w/w organic shea butter; (iii) 7.7% organic coconut oil; (iv) 30.9% organic peppermint flavoring agent; (v) 7.7% menthol; and (vi) 7.7% weight percentage 500 ppm colloidal silver, wherein the composition has an ability to function as a topically applied vehicle for ingredients and helps in treating fever blisters caused by the HSV-1 virus.

In yet another embodiment, the lip balm/ointment composition helps in healing of inflammatory reaction on lips.

In yet another embodiment, the composition reduces soreness, itchiness, and pain within 24 hours.

In yet another embodiment, the composition is solid at room temperature.

In yet another embodiment, the composition does not deteriorate under typical ambient temperatures.

In yet another embodiment, the lip balm/ointment composition is stored in containers wherein the containers have a volume from about 1 oz. to about 4 oz.

In yet another embodiment, the lip balm/ointment composition functions as a barrier against outside irritants like food and drinks.

In yet another embodiment, the lip balm/ointment composition has anti-microbial properties.

In yet another embodiment, a method of treating fever blisters caused by the HSV-1 virus in a human subject is described. The method includes the steps of topical application of a lip balm/ointment base composition on a cold sore area, the base composition comprising organic and natural ingredients consisting of beeswax, shea butter, liquid coconut oil, organic flavor (i.e. lemon, peppermint, et. al.), menthol, and/or colloidal silver wherein the method results in a reduced time to heal the fever blisters and cold sores as compared to conventional non-organic lip balm/ointment compositions.

Numerous benefits and advantages of this invention will become apparent to those skilled in the art to which it pertains upon reading and understanding of the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one potential embodiment of a small container storing the lip balm/ointment composition of the present invention in accordance with the disclosed architecture; and

FIG. 2 illustrates a perspective view of a user topically applying the lip balm/ointment composition of the present invention on a cold sore in accordance with the disclosed architecture.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there exists a long-felt need in the art for a composition that can be topically applied to blisters caused by HSV-1 for shortening healing time. There is also a long-felt need in the art for an improved lip balm/ointment composition that includes organic and natural ingredients and that can be used for healing cold sores. Additionally, there is a long-felt need in the art for a base composition that quickly reduces discomfort, pain, and redness of cold sore blisters. Moreover, there is a long-felt need in the art for a base composition that can be topically applied to the affected area on lips and adjacent skin, and can penetrate lip skin. Further, there is a long-felt need in the art for an improved lip balm/ointment that not only soothes the lips but also helps in quickly healing fever blisters caused by the HSV-1 virus. Finally, there is a long-felt need in the art for a base composition that offers a simple method of treating cold sores while significantly shortening healing time without using any harmful chemicals.

The present invention, in one exemplary embodiment, is a novel topical lip balm/ointment composition for topical application as an organic lip balm/ointment. The composition comprising a plurality of organic ingredients and is used for quickly healing of fever blisters caused by the HSV-1 virus. The composition consisting of (i) beeswax; (ii) shea butter; (iii) liquid coconut oil; (iv) organic flavoring agent; (v) menthol; and (vi) colloidal silver, each in amounts that are effective for collectively forming a topical base composition having an ability to topically apply as a lip balm/ointment, wherein the composition is effective in penetration of ingredients into lip skin for treating fever blisters caused by the HSV-1 virus.

The term “balm” as is used herein means a substance/composition that typically is oily or pasty in nature, a solid or semi-solid, and soothing to the skin of a human being when topically applied thereto. The “balm” may or may not include an aromatic compound having a pleasing taste and/or smell.

In accordance with the present invention, the topical composition/formulation of the lip balm/ointment for treating fever blisters caused due to the HSV-1 virus comprises only natural and organic ingredients. The topical composition is a stable formulation and remains solid at room temperature. The composition consists of beeswax, shea butter, liquid coconut oil, organic lemon or peppermint flavor, menthol, and colloidal silver. The lip balm/ointment can be used by a person for application to blisters without requiring a prescription from a medical doctor.

Beeswax is employed as a base ingredient and contains natural emulsifiers that helps retain moisture and prevents dry and chapped lips. Further, beeswax protects lips from harmful UV rays. The beeswax also functions as a softening agent, a thickening agent, and an emollient. The beeswax permits, or assists in permitting, the compositions of the invention to be formulated into a desired form and shape.

Beeswax is generally a colorless, nutrient-rich, liquid lipid that is secreted from special abdominal glands of honeybees. Beeswax is a complex substance made up of wax esters, fatty acids, and hydrocarbons. Beeswax typically contains from about 10 to about 15 percent paraffin carbohydrates, from about 35 to about 37 percent esters of C16 to C36 fatty acids, and about 15 percent cerotic acid, melissic acid, and their homologues. Beeswax typically is non-allergenic and includes water repellent properties. The amount of beeswax present in compositions of the lip balm/ointment ranges from about 10 weight percent to about 30 weight percent and preferably about 23 weight percent.

Shea butter is a great moisturizer that helps the balm to absorb and penetrate deep into lip skin. Shea butter remains solid at room temperature and thus helps the balm maintain its shape and consistency. Shea butter is a fat extracted from nuts of shea trees and may have an off-white or ivory color. High fat content of the shea butter functions as an excellent natural emollient and humectant, locking in moisture and keeping the lips and affected area hydrated. The amount of shea butter present in compositions of the lip balm/ointment ranges from about 10 weight percent to about 30 weight percent and preferably about 23 weight percent.

Organic coconut oil includes lauric acid that has nourishing properties and helps in healing seeping lip skin. Further, coconut oil provides vitamins and minerals and has a naturally pleasant scent. The coconut oil provides a beneficial remedy to heal chapped lips. The amount of coconut oil present in compositions of the lip balm/ointment ranges from about 5 weight percent to about 20 weight percent and preferably about 7.7 weight percent.

Menthol is an active ingredient used for pain relief. Menthol used in the composition is naturally derived from oil extracted from the leaves of the cornmint plant (Mentha arvensis) and provides a cooling sensation when applied to blisters. Further, menthol has pure natural flavor that helps in providing a refreshing taste. Menthol provides additional advantages of being vegan, diabetic friendly, and gluten free. Menthol is a waxy, clear or white crystalline substance, which is solid at room temperature and melts slightly above room temperature and thus helps in maintaining the shape and consistency of the composition at room temperature. The amount of menthol present in compositions of the lip balm/ointment ranges from about 2 weight percent to about 15 weight percent and preferably about 7.7 weight percent.

Organic flavoring agent is used for providing a pleasant flavor to the lip balm/ointment. Preferably, lemon or peppermint organic flavoring agents are used in the composition. The flavoring agents are alcohol-free, sugar-free, and add flavor to the balm. Natural peppermint flavor is a natural flavor, derived from mint leaves and is similar to spearmint. Natural organic lemon is made by extracting ingredients from perfectly ripe lemons and is not sour. The amount of flavoring agent present in compositions of the lip balm/ointment ranges from about 15 weight percent to about 35 weight percent and preferably about 30.9 weight percent.

Colloidal silver is an ingredient containing microscopic flakes of pure silver and functions as an antibacterial/antiviral agent, and a topical wound dressing. Colloidal silver inhibits HSV-1 infections by blocking the entrance of the virus into the cells and by preventing the cell-to-cell spread of the virus. More specifically, colloidal silver attaches to the cell walls of bacteria and viruses and damages their cell membrane to effectively heal cold sores quickly. The amount of colloidal silver present in compositions of the lip balm/ointment ranges from about 1 weight percent to about 40 weight percent and preferably about 7.7 weight percent.

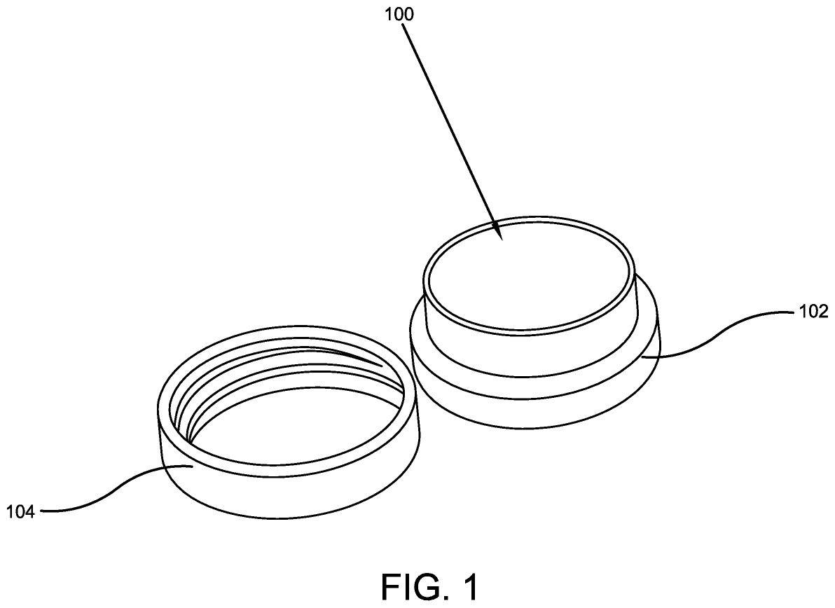

Referring initially to the drawings, FIG. 1 illustrates a perspective view of one potential embodiment of a small container storing the lip balm/ointment composition of the present invention in accordance with the disclosed architecture. The lip balm/ointment composition 100 of the present invention is solid at room temperature and has topical application for fever blisters caused by the HSV-1 virus. The composition 100 is commercially available in small containers 102 available in various sizes, such as, 1 oz., 2 oz., 3 oz., 4 oz., and more. The composition 100 can be wiped (i.e., removed) from the container 102 using a finger and can then be topically applied/transferred to an affected area. The composition 100 may change color over time due to the natural and organic ingredients. The container 102 may include the composition therein and a logo printed on an exterior surface of the container and also include a removable lid 104 for protecting the stored lip balm/ointment.

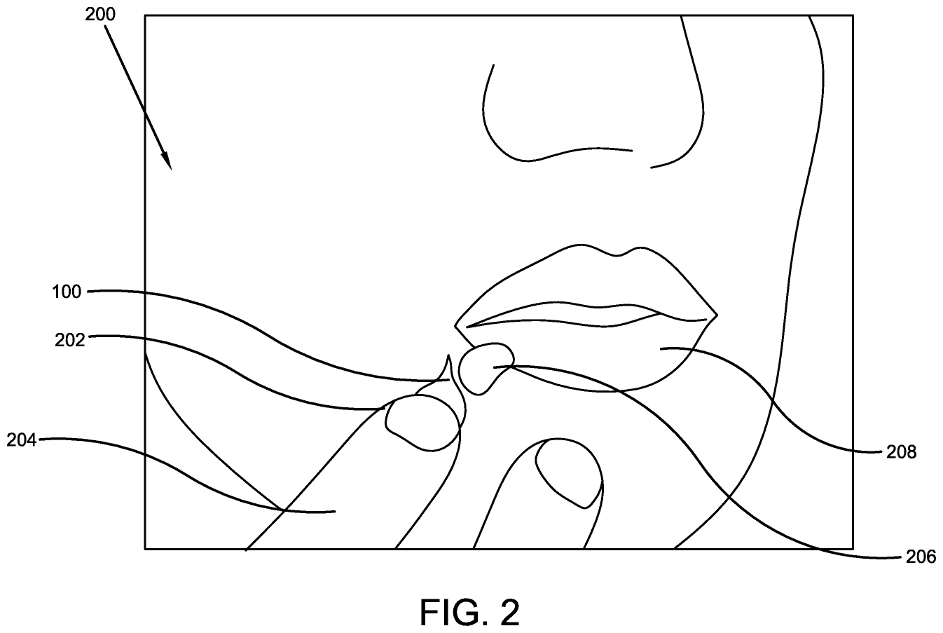

FIG. 2 illustrates a perspective view of a user topically applying the lip balm/ointment composition on a cold sore in accordance with the disclosed description. As illustrated, the user 200 takes a small quantity of the lip balm/ointment composition 100 from a tip 202 of a finger 204 and applies topically the lip balm/ointment to the cold sore 206. The lip balm/ointment 100 can be applied to the lips 208 and at the earliest onset of a cold sore 206 and can be applied multiple times a day on the lips 208 and the cold sore 206 (i.e., every 3-4 hours), or as needed, without any side effect.

It should be noted that the lip balm can be formed in the form of a stick which can be rolled on the affected area having a fever blister or a cold sore.

The lip balm compositions of the present invention provide the following advantages:
(i) penetration deep into the skin to the source of the spreading virus, blocking the virus, and providing a barrier to protect healthy skin cells;
(ii) lack of greasy or gritty feel but having smooth and non-sticky texture;
(iii) reliable and effective for individuals of all ages;
(iv) convenient to apply and administer;
(v) maintains shape and consistency over long periods of time and does not melt at room temperature; and
(vi) cosmetically appealing in appearance.

Table 1 below shows approximate concentrations of various ingredients in the lip balm composition.

TABLE 1

| | Composition in percentage |
|---|---|
| Ingredients | by weight (% w/w) |
| Beeswax | 10-30 |
| Organic shea butter | 10-30 |
| Menthol | 2-15 |
| Organic coconut oil | 5-20 |
| Organic lemon flavor | 15-35 |
| Colloidal silver | 1-40 |

Table 2 below shows approximate concentrations of various ingredients in another lip balm composition.

TABLE 2

| | Composition in percentage |
|---|---|
| Ingredients | by weight (% w/w) |
| Beeswax | 10-30 |
| Organic shea butter | 10-30 |
| Menthol | 2-15 |
| Organic coconut oil | 5-20 |
| Organic peppermint flavor | 15-35 |
| Colloidal silver | 1-40 |

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “base composition”, “lip balm composition”, “composition”, and “topical lip balm composition”, are interchangeable and refer to the cold sore healing topical lip balm composition 100 of the present invention.

Notwithstanding the foregoing, the cold sore healing topical lip balm composition 100 of the present invention can have any suitable natural ingredient as is known in the art without affecting the overall concept of the invention, provided that it accomplishes the above-stated objectives. One of ordinary skill in the art will appreciate that the composition of the cold sore healing topical lip balm composition 100 as shown in the FIGS. are for illustrative purposes only, and that many compositions are well within the scope of the present disclosure.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A topical lip balm composition comprising:
a beeswax;
a shea butter;
a liquid coconut oil;
an organic lemon flavor;
a menthol; and
a colloidal silver.

2. The topical lip balm composition of claim 1, wherein said beeswax comprises between 22 and 25 percent weight of the topical lip balm composition.

3. The topical lip balm composition of claim 1, wherein said shea butter comprises between 21 and 26 percent weight of the topical lip balm composition.

4. The topical lip balm composition of claim 1, wherein said liquid coconut oil comprises between 7 and 10 percent weight of the topical lip balm composition.

5. The topical lip balm composition of claim 4, wherein said organic lemon flavor comprises between 28 and 32 percent weight of the topical lip balm composition.

6. The topical lip balm composition of claim 5, wherein said menthol comprises between 5 and 9 percent weight of the topical lip balm composition.

7. The topical lip balm composition of claim 6, wherein a concentration of said colloidal silver is 500 ppm, and further wherein the colloidal silver comprises between 6 and 9 percent weight of the topical lip balm composition.

8. A topical lip balm composition comprising:
a beeswax;
a shea butter;
a liquid coconut oil;
an organic peppermint flavor;
a menthol; and
a colloidal silver.

9. The topical lip balm composition of claim 8, wherein said beeswax comprises between 10 and 30 percent weight of the topical lip balm composition.

10. The topical lip balm composition of claim 9, wherein said shea butter comprises between 10 and 30 percent weight of the topical lip balm composition.

11. The topical lip balm composition of claim 10, wherein said liquid coconut oil comprises between 5 and 20 percent weight of the topical lip balm composition.

12. The topical lip balm composition of claim 11, wherein said organic peppermint flavor comprises between 15 and 35 percent weight of the topical lip balm composition.

13. The topical lip balm composition of claim 12, wherein said menthol comprises between 2 and 15 percent weight of the topical lip balm composition.

14. The topical lip balm composition of claim 13, wherein a concentration of said colloidal silver is 500 ppm.

15. The topical lip balm composition of claim 14, wherein and colloidal silver comprises between 1 and 40 percent weight of the topical lip balm composition.

16. A method of treating a fever blister caused by an HSV-1 virus in a human subject, the method comprising the steps of:
combining a plurality of ingredients into a lip balm base composition, wherein said plurality of ingredients comprise a beeswax, a shea butter, a liquid coconut oil, an organic flavor, a menthol, and a colloidal silver; and
topically applying said lip balm base composition on the fever blister caused by the HSV-1 virus.

17. The method of claim 16, wherein said organic flavor is selected from a group consisting of a lemon flavor and a peppermint flavor.

18. The method of claim 17, wherein said beeswax comprises between 10 and 30 percent by weight of the lip balm base composition, wherein said shea butter comprises between 10 and 30 percent by weight of the lip balm base composition, wherein said liquid coconut oil comprises between 5 and 20 percent by weight of the lip balm base composition, wherein said organic flavor comprises between 15 and 35 percent by weight of the lip balm base composition, wherein said menthol comprises between 2 and 15 percent by weight of the lip balm base composition, and further wherein said colloidal silver comprises between 1 and 40 percent by weight of the lip balm base composition.

19. The method of claim 18, wherein said colloidal silver has a composition of 500 ppm.

20. The method of claim 19, wherein topically applying said lip balm composition includes a frequency of every 3 to 4 hours.
